(19) United States
(12) Patent Application Publication
(10) Pub. No.: US 2005/0281855 A1
(43) Pub. Date: Dec. 22, 2005

(54) Process for preparing a cross-linked carboxyl polysaccharide and the cross-linked carboxyl polysaccharide

(75) Inventor: Jenn-Jong Young, Chungho City (TW)

Correspondence Address: NIKOLAI & MERSEREAU, P.A., 900 SECOND AVENUE SOUTH, SUITE 820, MINNEAPOLIS, MN, 55402, US

(73) Assignee: National Defense Medical Center, Taipei (TW)

(21) Appl. No.: 10/872,245

(22) Filed: Jun. 18, 2004

(51) Int. Cl. A61K 38/19; A61K 38/20; A61K 39/395; A61K 38/21

(52) U.S. Cl. 424/422; 536/21; 536/53; 536/54; 424/85.1; 424/85.4; 424/130.1

(57) ABSTRACT

A process for preparing a cross-linked polysaccharide comprises providing a polysaccharide with free carboxyl and hydroxyl groups capable of forming an intermolecular ester bond and cross-linking the polysaccharide by using onium salt, phosphonium salt, uronium salt or carbenium salt and in the presence or in the absence of organic base as cross-link reagent to obtain a highly cross-linked polysaccharide. The cross-linked polysaccharide has high cross-linking density and is stable and slowly biodegradable.

### BACKGROUND OF THE INVENTION

1. Field of the Invention

The present invention relates to a process for preparing a cross-linked carboxyl polysaccharide on a heterogeneous reaction condition and the product prepared by the process, and more particularly to a process for preparing a stable biocompatible and highly cross-linked carboxyl polysaccharide in different physical forms by using onium salt, phosphonium salt, uronium salt or carbenium salt, in the presence or in the absence of organic base, as cross-link reagent on a heterogeneous reaction condition.

2. Description of Related Art

Since biopolymers are biocompatible and biodegradable, biopolymers are used widely in artificial dressings, scaffold for tissue engineering, drug releasing media, orthopedics, dental devices and cosmetics. The usual natural biopolymers used include proteins, peptides or polysaccharides.

In principle, directly using a natural biopolymer is limited by the characteristics of the biopolymer, for example, water solubility, the short retention time in tissue, the quick absorption in the tissue, etc. To overcome the inherent characteristics of the biopolymer, different reagents (e.g. formaldehyde, glutaraldehyde, divinyl sulfone, phosphoryl chloride, diglycidyl ether, dihydrazide and ethylenediamine) or natural cross-link reagents (e.g. genipin and reuterin) have been used for cross-linking and construct water-insoluble and highly stable biopolymers with a 3-D net work structure. However, the foregoing reagents may themselves be toxic chemicals and chemically bind to biopolymers, and the stabilized three-dimensional matrix may not have the same degree of biological activity as the flexible water-soluble molecule.

Auto-cross-linked polysaccharides (ACP) are a new class of carboxyl polysaccharide derivatives obtained through an inter- and intramolecular esterification of polysaccharides in which part of the carboxyl group is esterified with a hydroxyl group of the same and/or different molecules of polysaccharide.

2-chloro-1-methylpyridinium iodide (CMPI) has been used as a cross-link reagent to cross-link hyaluronic acid (HA) tetrabutylammonium salt in dimethyl sulfoxide or N-methylpyrrolidone solution to produce ACP in a solid form. However, the reaction must be performed on HA quaternary ammonium salt, obtained by ion displacement technique from HA sodium salt, and in organic solvent on a homogeneous reaction condition, the starting materials are hardly obtained and the purification procedure is long-winded and inconvenient.

Water-soluble carbodiimide (WSC) such as 1-ethyl-3-(3-dimethylamino propyl) carbodiimide hydrochloride (EDC) has been used as a cross-link reagent to cross-link HA film using the film immersion method. EDC also used as a reagent to produce cross-linked gelatin-alginate sponge and gelatin-hyaluronate sponge on a heterogeneous reaction condition. Although these EDC cross-lining reactions succeeded in making HA film water insoluble, the resultant cross-linked films were dissolved within a few days in a phosphate buffered saline (PBS) solution of pH 7.4 at 37° C.

The conventional methods can not prepare a highly cross-linked carboxyl polysaccharides and the resultant cross-linked carboxyl polysaccharides is unstable biocompatible and dissolved completely in PBS at 37° C. within a few days.

### SUMMARY OF THE INVENTION

An aspect of the present invention is to provide a facile process for preparing a cross-linked carboxyl polysaccharide comprising providing a polysaccharide with free carboxyl and hydroxyl groups capable of forming an intermolecular ester bond on a heterogeneous reaction condition and using onium salt, phosphonium salt, uronium salt or carbenium salt as a cross-link reagent in the presence or in the absence of organic base to obtain a highly cross-linked carboxyl polysaccharide in a different physical form.

Another aspect of the present invention is to provide a cross-linked carboxyl polysaccharide prepared by the foregoing process. The cross-linked carboxyl polysaccharide film produced with the foregoing cross-linking reaction has high cross-linking density, is stable and slowly biodegradable in the presence of hydrolysis enzyme and retains 80% of its original weight after standing in PBS (pH 7.4) at 37° C. for at least four weeks.

Further benefits and advantages of the present invention will become apparent after a careful reading of the detailed description.

### BRIEF DESCRIPTION OF THE DRAWING

FIG. 1 shows in vitro degradation of CMPI or EDC-cross-linked HA films in PBS Hyaluronidase (200 units/ml) or PBS solution at 37° C.

### DETAILED DESCRIPTION OF THE INVENTION

A preferred embodiment of a process for preparing a cross-linked carboxyl polysaccharide in accordance with the present invention comprises providing a polysaccharide with free carboxyl and hydroxyl groups capable of forming an intermolecular ester bond on a heterogeneous reaction condition and cross-linking the polysaccharide by using onium salt, phosphonium salt, uronium salt or carbenium salt as a cross-link reagent in the presence or in the absence of organic base to obtain a highly cross-linked polysaccharide in a different physical form.

The carboxyl polysaccharide preferably used in the present invention maybe include hyaluronic acid, alginic acid, pectin, heparin, heparin sulphate, chondroitin sulphate, dermatan sulphate, keratan sulphate, keratosulphate, branan ferulate, a derivative of the foregoing and a combination thereof.

Furthermore, the carboxyl polysaccharide preferably used in the present invention on a heterogeneous reaction condition maybe in the form of a film, a sponge, a gel, a hydrogel, a microsphere, a bead, a fiber or a nanoparticle.

The onium salt preferably used in the present invention maybe includes 2-halogen-N-alkyl pyridinium, pyrimidinium, benzoxazolium, benzothiazolium salt, in which the halogen is selected from the group consisting of chlorine and bromine and the alkyl has a maximum of 6 carbon atoms, a derivative of the foregoing and a combination thereof.

The phosphonium salt preferably used in the present invention maybe include (benzotriazol-1-yloxy)tripyrrolidinophosphonium salt (PyBOP), (benzotriazol-1-yloxy)tris(dimethylamino)phosphonium salt (BOP), μ-oxo-bis[tris(dimethylamino)phosphonium] bis-salt (Bates reagent), a derivative of the foregoing and a combination thereof.

The uronium or carbenium salt preferably used in the present invention maybe include O-(benzotriazol-1-yl)-N,N,N′,N′-bis(tetra-methylene)uronium salt ((benzotriazol-1-yloxy)dipyrrolidino carbenium salt) (HBPyU), O-(benzotriazol-1-yl)-N,N,N′,N′-bis(pentamethylene)uronium salt ((benzotriazol-1-yloxy)dipiperidinocarbenium salt) (HBPipU), O-(benzotriazol-1-yl)-N,N,N′,N′-tetramethyluronium salt (HBTU), chlorodipyrrolidino-carbenium salt (CDPC), a derivative of the foregoing and a combination thereof.

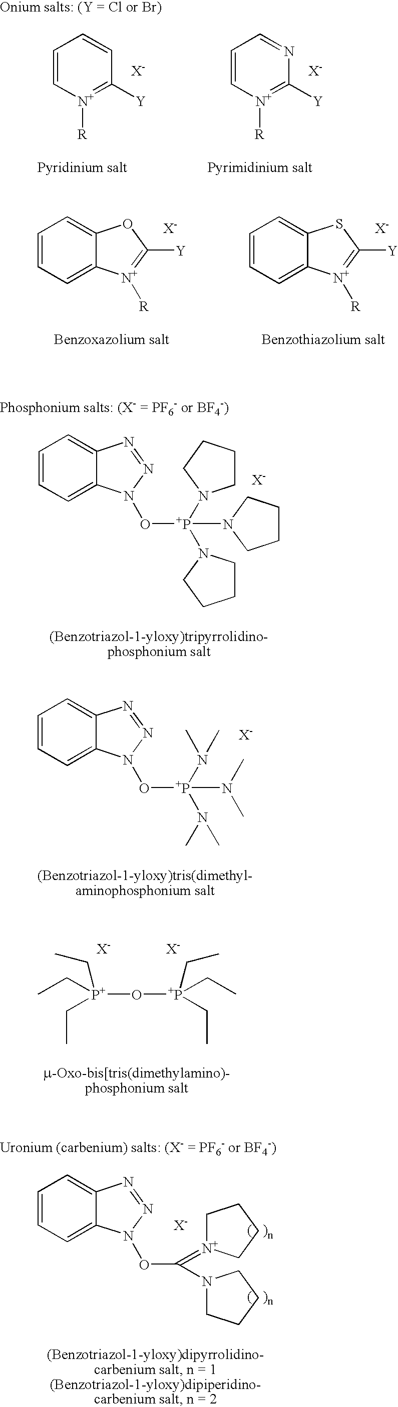

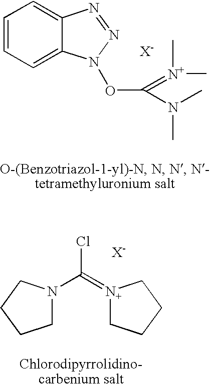

Preferably, the organic base used in the present invention maybe include tertiary amine less than 20 carbon atoms, pyridine, 4-dialkylaminopyridine, 1,5-diazabicyclo[4.3.0]non-5-ene, 1,8-diazabicyclo[5.4.0]undec-7-ene, 1,4-diazabicyclo[2.2.2]octane, a derivative of the foregoing and a combination thereof.

Preferably, the cross-linking reaction of onium salt, phosphonium salt, uronium salt or carbenium salt is carried out in an aqueous solution at a temperature of 0 to 150° C. Preferably, the aqueous onium salt, phosphonium salt, uronium salt or carbenium salt solution is present at a concentration in the range between 1 mM and 1M.

Preferably, the molar equivalent ratio of the onium salt, phosphonium salt, uronium salt or carbenium salt to the carboxyl polysaccharide (base on the carboxyl groups) is at least 1:100.

Preferably, the molar equivalent ratio of the organic base to the carboxyl polysaccharide (base on the carboxyl groups) is present in the range of less than 10.

Preferably, the aqueous solution used in the present invention maybe include the mixture of protic or aprotic solvent and water in the range between 1/99 and 99/1. More preferably, the protic or aprotic solvent used in the present invention maybe include alcohol, ketone or ether less than 10 carbon atoms, tetrahydrofuran, dioxane, dimethylsulfoxide, N,N-dimethylformamide, acetonitrile, a derivative of the foregoing and a combination thereof.

Preferably, the cross-linked carboxyl polysaccharide used in the present invention maybe in the form of a film, a sponge, a gel, a hydrogel, a microsphere, a bead, a fiber or a nanoparticle.

In a preferred embodiment of the present invention, a biomedical material comprises the highly cross-linked carboxyl polysaccharide as described foregoing.

Preferably, the biomedical material is for use as a scaffold for cell growth in tissue engineering or as an implant or a component of an implant.

Preferably, the implant used in the present invention maybe capable of releasing cytokines, growth factors, peptides, enzymes, drugs, immunogens or antibodies.

When using carbodiimide as a cross-link reagent to carry out a HA cross-linking reaction, carbodiimide reacts with the carboxyl group of the HA to form an unstable intermediate O-acylurea. An acidic environment is needed to catalyze the reaction, presumably through the protonation of the carbodiimide nitrogen. At pH 4.75, carbodiimide nitrogen appears to be sufficiently protonated, while HA mainly exists as the carboxylate. The proton is not only a catalyst. One proton is consumed to form the O-acylurea, thus the pH increases during the reaction process. Under basic conditions, the intermediate O-acylurea quickly rearranges to form a stable N-acylurea by means of an O→N migration mechanism. Since the present invention didn't add any acid to adjust the pH during the heterogeneous cross-linking reaction process, the activation rate decreased and finally stopped as the proton was consumed. Thus, only a few of the carboxyl groups were chemically transferred into O-acylurea. The O-acylurea showed a relatively low reactivity, it quickly rearranged to N-acylurea or leaved as unreactive O-acylurea and only a few of the ester bonds were formed. Introduction of the hydrophobic acylurea substituents into the carboxyl group of HA film can also reduce a lot of its water uptake ability and water solubility, such as: ethyl or benzyl ester of HA. However, there are little effects on the stability and degradation rate because only a few of the cross-linking bonds were formed to construct the three-dimensional networks.

When using an onium salt, phosphonium salt, uronium salt or carbenium salt such as CMPI, BOP, PyBOP, HBPyU, HBPipU, HBTU, CDPC as the cross-link reagent to carry out the HA cross-linking reaction, it activates the carboxyl group of the HA to form an intermediate. The intermediate will not occur to rearrange, and the intermediate shows a relatively high reactivity and may react with the hydroxyl group of the same and/or different molecules of HA to form an inter- and/or intramolecular esterification. However, the preceding reaction will release a proton to decrease the reaction rate. Because of this condition, an organic base must be added to sustain a cross-linking reaction in some cases.

The pH plays an important role in the cross-linking reaction. In an embodiment of the present invention, using the onium salt, phosphonium salt, uronium salt or carbenium salt as a cross-link reagent in the presence or in the absence of organic base will generate a facile heterogeneous cross-linking system to produce a high cross-linking degree of carboxyl polysaccharide in a different physical form which is stable and slowly biodegradable in the presence of hydrolysis enzyme. Preferably, the resultant product retains 80% of its original weight after standing in PBS (pH 7.4) at 37° C. for four weeks. The highly cross-linked carboxyl polysaccharide can be applied in scaffolds for tissue engineering, wound healing, ophthalmic surgery, arthritis treatment and the components of implant materials.

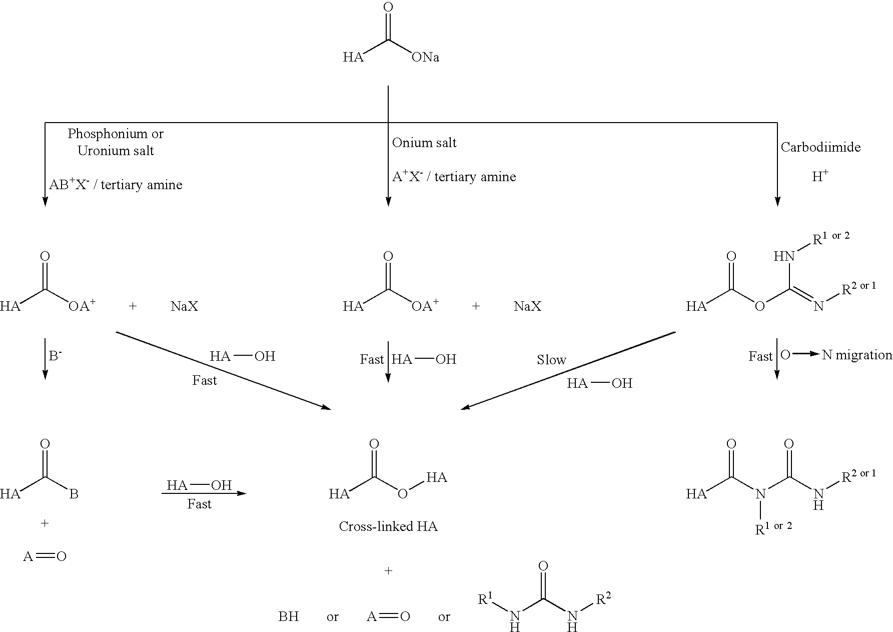

Further details of this invention are illustrated in the following examples.

### EXAMPLE 1

Preparation of Cross-Linked HA Film by CMPI

HA (2 wt %) in aqueous solution was prepared from HA powder using distillated water. Then, 30 g of viscous HA solution was poured into a petri dish (diameter 8.6 cm). The cast solution was allowed to air dry at room temperature, and then the film was peeled off and dried in vacuo (<0.1 mmHg) over 8 hours before being further used.

HA film was weighed and directly immersed in an ethanol/water mixture (8:2 v/v) containing 0.4 equivalents (molar ratios of reagent based on the carboxylate groups in HA) of 10 mM CMPI, then shaken at room temperature for three days. The cross-linked film was washed with 80% ethanol three times (3×50 mL) and placed between two pieces of filter paper over night to flatten it out. The film was dried in vacuo (<0.1 mmHg) for at least 8 hours before being further used.

### EXAMPLE 2

In Vitro Degradation of CL-HA Film

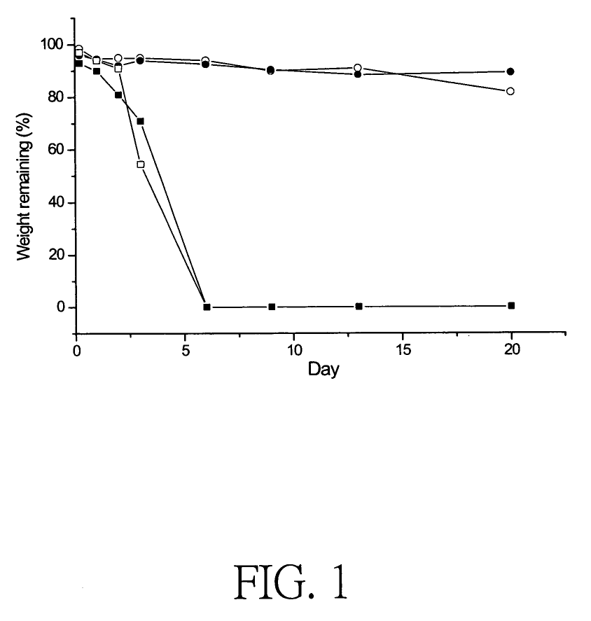

Pieces of CMPI-CL-HA (from example 1) or EDC-CL-HA films with known dry weights were immersed in PBS HAse (200 units/ml) solution or PBS solution at 37° C. The swollen films were taken out at predetermined days and washed with water three times. The swollen films were dried in vacuo (<0.1 mmHg) at room temperature for 16 hours and then weighed again to determine the percentage of weight remaining by the equation (1).
Weight remaining (%)=Wd/Wo×100 (1)

Where Wo is the weight before degradation and Wd is the weight after degradation. The weight remaining permits to estimate the in vitro enzymatic degradation (FIG. 1). (∘) CMPI-CL-HA in PBS HAse; (●) CMPI-CL-HA in PBS; (□) EDC-CL-HA in PBS HAse; (▪) EDC-CL-HA in PBS.

### EXAMPLE 3

Preparation of Cross-Linked HA Film by CDPC

HA film was weighed and directly immersed in an acetone/water mixture (8:2 v/v) containing 1 equivalents (molar ratios of a reagent based on the carboxylate groups in HA) of 25 mM CDPC and in the presence of 2 equivalents of triethylamine, then shaken at room temperature for three days. The cross-linked film was washed with 80% acetone three times (3×50 mL) and placed between two pieces of filter paper over night to flatten it out. The film was dried in vacuo (<0.1 mmHg) for at least 8 hours before being further used.

### EXAMPLE 4

Preparation of Cross-Linked HA Sponge by CMPI

30 g of HA (2 wt %) viscous solution was poured into a petri dish (diameter 8.6 cm). The solution was frozen at −35° C., lyophilized at −35° C. for 3 days and resulted in a primrose yellow HA sponge.

HA sponge was weighed and directly immersed in an ethanol/water mixture (8:2 v/v) containing 1 equivalents (molar ratios of a reagent based on the carboxylate groups in alginate) of 25 mM CMPI and triethylamine, then shaken at room temperature for three days. The cross-linked sponge was washed with 80% ethanol three times (3×50 mL), and 20 mL water was added to make the cross-linked sponges absorb the water and swell. The sponge was lyophilized at −35° C. for 3 days and resulted in a primrose yellow cross-linked HA sponge.

### EXAMPLE 5

Preparation of Cross-Linked HA/Alginate (1:1) Film by CMPI

25 g of a 2 wt % HA/alginate (1:1 w/w) viscous solution was poured into a petri dish (diameter 8.6 cm). The solution was allowed to air dry at room temperature, and then the film was peeled off and dried in a vacuum (<0.1 mmHg) over 8 hours before being further used.

HA/alginate (1:1 w/w) film was weighed and directly immersed in an ethanol/water mixture (8:2 v/v) containing 1 equivalents (molar ratios of a reagent based on the carboxylate groups in HA and alginate) of CMPI at a concentration of 30 mM, then shaken at room temperature for three days. The cross-linked film was washed with 80% ethanol three times (3×50 mL) and placed between two pieces of filter paper over night to flatten it out. The film was dried in vacuo (<0.1 mmHg) for at least 8 hours before being further used.

Although the invention has been explained in relation to its preferred embodiment, many other possible modifications and variations can be made without departing from the spirit and scope of the invention as hereinafter claimed.

1. A process for preparing a highly cross-linked carboxyl polysaccharide on a heterogeneous reaction condition comprising:
providing a polysaccharide with free carboxyl and hydroxyl groups capable of forming an intermolecular ester bond; and
cross-linking the carboxyl polysaccharide by using onium salt, phosphonium salt, uronium salt or carbenium salt as cross-link reagent in the presence or in the absence of organic base to obtain a highly cross-linked carboxyl polysaccharide.

2. The process as claimed in claim 1, wherein the carboxyl polysaccharide is selected from the group consisting of hyaluronic acid, alginic acid, pectin, heparin, heparin sulphate, chondroitin sulphate, dermatan sulphate, keratan sulphate, keratosulphate, branan ferulate, a derivative of the foregoing and a combination thereof.

3. The process as claimed in claim 1, wherein the carboxyl polysaccharide is in the form of a film, a sponge, a gel, a hydrogel, a microsphere, a bead, a fiber or a nanoparticle.

4. The process as claimed in claim 1, wherein the onium salt is selected from the group consisting of 2-halogen-N-alkyl pyridinium, pyrimidinium, benzoxazolium, benzothiazolium salts, in which the halogen is selected from the group consisting of chlorine and bromine and the alkyl has a maximum of 6 carbon atoms, a derivative of the foregoing and a combination thereof.

5. The process as claimed in claim 1, wherein the phosphonium salt is selected from the group consisting of (benzotriazol-1-yloxy)tripyrrolidino-phosphonium salt (PyBOP), (benzotriazol-1-yloxy)tris(dimethylamino)-phosphonium salt (BOP), μ-oxo-bis[tris(dimethylamino)phosphonium] bis-salt (Bates reagent), a derivative of the foregoing and a combination thereof.

6. The process as claimed in claim 1, wherein the uronium or carbenium salt is selected from the group consisting of O-(benzotriazol-1-yl)-N,N,N′,N′-bis(tetramethylene)uronium salt ((benzotriazol-1-yloxy) dipyrrolidinocarbenium salt) (HBPyU), O-(benzotriazol-1-yl)-N,N,N′,N′-bis(pentamethylene)uronium salt ((benzotriazol-1-yloxy)dipiperidinocarbenium salt) (HBPipU), O-(benzotriazol-1-yl)-N,N,N′,N′-tetramethyluronium salt (HBTU), chlorodipyrrolidinocarbenium salt (CDPC), a derivative of the foregoing and a combination thereof.

7. The process as claimed in claim 1, wherein the organic base is selected from the group consisting of tertiary amine less than 20 carbon atoms, pyridine, 4-dialkyllaminopyridine, 1,5-diazabicyclo[4.3.0]non-5-ene, 1,8-diazabicyclo[5.4.0]undec-7-ene, 1,4-diazabicyclo[2.2.2]octane, a derivative of the foregoing and a combination thereof.

8. The process as claimed in claim 1, wherein the molar equivalent ratio of the onium salt, phosphonium salt, uronium salt or carbenium salt to the carboxyl polysaccharide (base on the carboxyl groups) is at least 1:100.

9. The process as claimed in claim 1, wherein the molar equivalent ratio of the organic base to the carboxyl polysaccharide base on the carboxyl group is in the range of less than 10.

10. The process as claimed in claim 1, wherein the cross-linking reaction of onium salt, phosphonium salt, uronium salt or carbenium salt is carried out in an aqueous solution at a temperature of 0 to 150° C.

11. The process as claimed in claim 10, wherein the aqueous solutions is present at a concentration in the range between 1 mM and 1M.

12. The process as claimed in claim 10, wherein the aqueous solutions is a mixture of aprotic or protic solvent and water in the range between 1/99 and 99/1.

13. The process as claimed in claim 12, wherein the aprotic or protic solvent is selected from the group consisting of alcohol, ketone or ether less than 10 carbon atoms, tetrahydrofuran, dioxane, dimethylsulfoxide, N,N-dimethylformamide, acetonitrile, a derivative of the foregoing and a combination thereof.

14. A cross-linked carboxyl polysaccharide prepared by the process as claimed in claim 1.

15. The cross-linked carboxyl polysaccharide as claimed in claim 14 in the form of a gel, a film, a sponge, a hydrogel, a microsphere, a fiber, a bead or a nanoparticle.

16. A biomedical material comprising the highly cross-linked carboxyl polysaccharide as claimed in claim 14.

17. The biomedical material as claimed in claim 16 for use as a scaffold for cell growth in tissue engineering.

18. The biomedical material as claimed in claim 16 for use as an implant or a component of an implant.

19. The biomedical material as claimed in claim 18, wherein the implant is capable of releasing cytokines, growth factors, peptides, enzymes, drugs, immunogens or antibodies.
